(19) United States
(12) Patent Application Publication
(10) Pub. No.: US 2025/0051377 A1
(43) Pub. Date: Feb. 13, 2025

(54) SULFONATE ESTERIFIED PHOSPHAZENE COMPOUNDS

(71) Applicant: SABIC Global Technologies B.V., Bergen op Zoom (NL)

(72) Inventors: Tianhua Ding, Bengaluru (IN); Yunyang Liu, Bengaluru (IN)

(21) Appl. No.: 18/723,579

(22) PCT Filed: Dec. 23, 2022
(86) PCT No.: PCT/EP2022/087696

§ 371 Date: Jun. 24, 2024

(30) Foreign Application Priority Data
Dec. 27, 2021 (EP) 21217769.5

(51) Int. Cl. C07F 9/6593; C07C 315/04; C08K 5/5399; C08L 69/00

(52) U.S. Cl. CPC C07F 9/65815; C07C 315/04; C08K 5/5399; C08L 69/00; C08L 2201/02; C08L 2205/025

(57) ABSTRACT

The invention is directed to sulfonate esterified phosphazene compounds, which include cyclic, linear, or cross-linked, phosphazene compounds. The invention further relates to methods of preparing such sulfonate esterified phosphazene compounds and to polymer compositions comprising the phosphazene compounds. The invention also relates to articles comprising such polymer compositions and to the use of such sulfonate esterified phosphazene compounds for improving the fire retardancy properties of polymer compositions.

### CROSS REFERENCE TO RELATED APPLICATIONS

This application is a National Stage Application under 35 U.S.C. § 371 and claims the benefit of International Application No. PCT/EP2022/087696, filed Dec. 23, 2022, which claims benefit to European Patent Application No. EP 21217769.5, filed Dec. 27, 2021, the contents of both of which are incorporated herein by reference in their respective entirety.

### FIELD

The present disclosure relates to the field of phosphazene compounds and more particularly to sulfonate esterified phosphazene compounds suitable as fire retardants in polymeric formulations. The disclosure further relates to methods of preparing such sulfonate esterified phosphazene compounds and to polymer compositions comprising the phosphazene compounds. The disclosure also relates to articles comprising such polymer compositions and to the use of such sulfonate esterified phosphazene compounds for improving the fire retardancy properties of polymer compositions.

### BACKGROUND

Flame resistance (FR) additives are commonly added to polymer products used in construction, automotive, electronic, electrical laminate, wire and cable, textile and other applications. FR additives increase the limiting oxygen index (LOI) of polymer systems, allowing articles made from those polymer systems to pass standard fire tests. Various low molecular weight (<˜1500 g/mol) brominated compounds are used as FR additives for organic polymers. Many of these, such as hexabromocyclododecane and polybrominated diphenylethers, are under regulatory and public pressure that may lead to restrictions on their use, and therefore there is an incentive to find a replacement for them.

Previously, various phosphorus compounds have been used as FR additives. These include organic phosphates, phosphonates and phosphoramides, some of which are described in U.S. Pat. Nos. 4,070,336 and 4,086,205, as well as in “The Chemistry and Use of Flame Retardants”, J. W. Lyons, Chapter 2-Chemistry of Fire Retardants Based on Phosphorous p.29-74 (1987). These compounds tend to provide moderate ignition resistance, and are generally not as effective as hexabromocyclododecane or other brominated FR additives.

Phosphazenes are a class of phosphorus compounds that contain a chain of alternating phosphorus and nitrogen atoms. Phosphazenes can be linear or cyclic in structure. Certain compounds of both types have been tried as FR additives in specific polymer systems. U.S. Pat. No. 3,994,996 describes compounds having multiple cyclic phosphazene moieties that are bonded together through P—O—P bonds. Those compounds are said to be useful FR additives for rayon. In U.S. Pat. No. 4,864,047, aminophenoxycyclotriphosphazenes are said to be useful as flame retardants for polystyrene. JP 2004-051697A describes certain other cyclic phosphazenes as FR additives for a variety of polymers, including polyolefins, polystyrene and styrene copolymers, polycarbonates, poly (ethylene terephthalate), polybutadiene, polyamides, epoxy resins and unsaturated polyesters, as well as others. These are described as flame retardants for a variety of polymers, similar to those described in JP 2004-051697. The patent applications JP 2001-200151A and U.S. Published Application No. 2005-0245670 all describe certain substituted cyclic phosphazenes as FR additives in polycarbonate resin compositions.

Despite numerous attempts such as these, phosphazene compounds have struggled to find commercial success. Although phosphazene compounds can provide flame retardancy (as indicated by certain standardized tests), they often do not have other attributes that are necessary in a good FR additive. In some cases, the phosphazene compounds are simply too expensive to be practical. Many of the phosphazene FR additives have been found to be inefficient, and require somewhat large loadings in order to be effective. Sometimes, the phosphazene compounds are not sufficiently miscible in particular organic polymers. As a result of this, it is difficult to distribute the phosphazene compound uniformly into the polymer. This leads to inconsistent performance and in some instances the loss of the FR additive from the polymer over time, due to bleeding or other processes. Poor miscibility can also lead to serious processing problems. In some cases, an effective amount of the phosphazene compound cannot be introduced into the polymer because of poor miscibility.

Yet another problem with some phosphazene FR additives is that they are not thermally stable at the temperatures at which certain organic polymers are melt-processed. This is particularly the case when the polymer is melt processed at temperatures above 230° C. It has been observed that many of the phosphazene compounds begin to degrade at those temperatures, leading to the formation of decomposition products and, of course, loss of the additive.

Thus, for the foregoing reasons, there remains a need for developing a phosphazene compounds capable of providing excellent flame retardancy properties at lower loadings in polymeric formulations along with excellent thermal stability, suitable for being processed with polymer melts.

### BRIEF SUMMARY

Accordingly, a solution to some or all of the drawbacks in existing art, resides in a sulfonate esterified phosphazene compound selected from the group consisting of:
i. a cyclic phosphazene compound represented by the formula 1a:

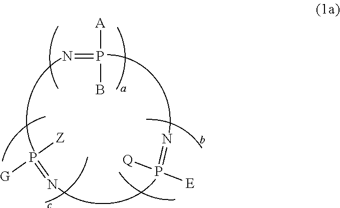

wherein each of the variables ‘a’, ‘b’ and ‘c’ are integers ranging from ≥1 and ≤5; preferably each of the variable ‘a’, ‘b’ and ‘c’ is 1;
ii. a linear phosphazene compound represented by the formula (1b):

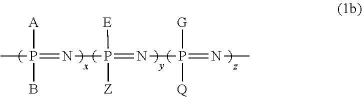

wherein each of the variables ‘x’, ‘y’ and ‘z’ are integers ranging from ≥1 and ≤500, preferably from ≥50 and ≤200, preferably from ≥100 and ≤200;
iii. a cross-linked phosphazene compound represented by the formula (1c):

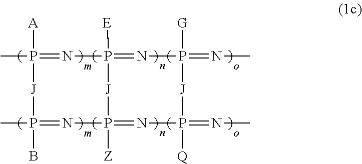

wherein each of the variables ‘m’, ‘n’ and ‘o’ are integers ranging from ≥1 and ≤500, preferably from ≥50 and ≤200, preferably from ≥100 and ≤200;
wherein each of the substituents ‘A’, ‘B’, ‘E’, ‘Z’, ‘G’, ‘Q’, ‘J’ are independently same or different, wherein each of the substituents ‘A’, ‘B’, ‘E’, ‘Z’, ‘G’, ‘Q’, ‘J’ are independently selected from a substituted or an unsubstituted alkyl group having 1-20 carbon atoms, a branched or a linear alkyl group having 1-20 carbon atoms, an alkenyl group having 1-20 carbon atoms, an ether group having 1-20 carbon atoms, an alkoxy group having 1-20 carbon atoms, an aryl group having 6-30 carbon atoms, an alkaryl group having 6-30 carbon atoms, an aralkyl group having 6-30 carbon atoms, a cycloalkyl group having 3-10 carbon atoms, a substituted or an unsubstituted amine group, and a substituted sulfonate esterified group represented by the formula (1d):

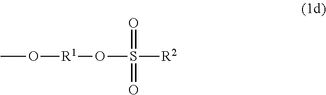

wherein R¹ is a substituent selected from a group comprising at least one aryl group comprising 6-30 carbon atoms, preferably 8-30 carbon atoms, preferably 8-20 carbon atoms, preferably 10-18 carbon atoms, preferably 11-18 carbon atoms; wherein R² is a substituent selected from a group consisting of a halogen substituted or an unsubstituted alkyl group having 1-20 carbon atoms, a branched or a linear alkyl group having 1-20 carbon atoms, a halogen substituted aryl group, a hydroxyl substituted aryl group, a nitro group substituted aryl group, an alkaryl group having 6-30 carbon atoms, an aralkyl group having 6-30 carbon atoms, a cycloalkyl group having 3-10 carbon atoms; with the proviso that at least one of the substituent ‘A’, ‘B’, ‘E’, ‘Z’, ‘G’, ‘Q’ is a sulfonate esterified group as represented by the formula (1d), preferably wherein each of the substituent ‘A’, ‘B’, ‘E’, ‘Z’, ‘G’, ‘Q’ is a sulfonate esterified group as represented by the formula (1d).

In some embodiments of the disclosure, the disclosure is directed to a method of preparing a sulfonate esterified phosphazene compound, comprising the steps of:
i. reacting a diol compound (V) represented by the formula

HO—R¹—OH
with a sulfonyl halide compound represented by the formula (W)

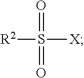

and forming a first intermediate compound; wherein the substituents R¹ and R² are as defined in herein throughout this disclosure; wherein the substituent ‘X’ is a halogen; preferably wherein the substituent ‘X’ is chlorine;
ii. reacting the first intermediate compound with a phosphazene compound (T) having at least one phosphorous atom substituted by a nucleophilic leaving group (NLG), and forming a sulfonate esterified phosphazene compound;
   wherein the nucleophilic leaving group (NLG) is selected from the group consisting of a halogen group, a thio group, a cyano group, a tosylate group, an alkoxy group, an aryloxy group, an alkyl ester group, an azide group, an amine group, preferably the nucleophilic leaving group (NLG), is a halogen group.

In some embodiments of the disclosure, the disclosure is directed to a polymer composition comprising:
i. ≥80.0 wt. % and ≤99.95 wt. %, preferably ≥85.0 wt. % and ≤99.95 wt. %, with regard to the total weight of the composition, of a thermoplastic polymer; and
ii. ≥0.05 wt. % and ≤20.0 wt. %, preferably ≥0.05 wt. % and ≤15.0 wt. %, with regard to the total weight of the composition, of the sulfonate esterified phosphazene compound of the present disclosure.

Other objects, features and advantages of the present disclosure will become apparent from the following figures, detailed description, and examples. It should be understood, however, that the figures, detailed description, and examples, while indicating specific embodiments of the disclosure, are given by way of illustration only and are not meant to be limiting.

Additionally, it is contemplated that changes and modifications within the spirit and scope of the disclosure will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### DETAILED DESCRIPTION

The following includes definitions of various terms and phrases used throughout this specification.

Any numerical range used throughout this disclosure shall include all values and ranges there between unless specified otherwise. For example, a boiling point range of 50° C. to 100° C. includes all temperatures and ranges between 50° C. and 100° C. including the temperature of 50° C. and 100° C.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.” The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The process of the present disclosure can “comprise”, “consist essentially of,” or “consist of” particular ingredients, components, compositions, etc., disclosed throughout the disclosure.

Any formula or structure containing a carbon atom, which is not denoted expressly by a specific substituent may be construed to include any one of a hydrogen substituent, or a hydrocarbyl substituent, or a heteroatom substituent, or an amine substituent.

The disclosure addresses the need for developing non-halogen fire retardants such as phosphazene compounds, which are capable of providing excellent flame retardancy properties at lower loadings in polymeric formulations along with excellent thermal stability and suitable for being processed with polymer melts.

The phosphazene compound is a sulfonate esterified phosphazene compound. Preferably such sulfonate esterified phosphazene compounds may be a cyclic phosphazene compound or a linear phosphazene compound or a cross-linked phosphazene compound.

In some preferred embodiments of the disclosure, the sulfonate esterified phosphazene compound is a cyclic phosphazene compound represented by the formula (1a):

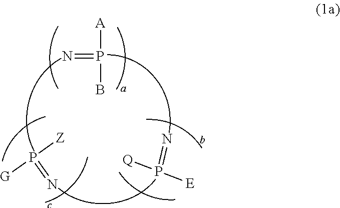

wherein each of the variables ‘a’, ‘b’ and ‘c’ are integers ranging from ≥1 and ≤5, wherein each of the substituents ‘A’, ‘B’, ‘E’, ‘Z’, ‘G’, ‘Q’, are independently same or different, wherein each of the substituents ‘A’, ‘B’, ‘E’, ‘Z’, ‘G’, ‘Q’, are independently selected from a substituted or an unsubstituted alkyl group having 1-20 carbon atoms, a branched or a linear alkyl group having 1-20 carbon atoms, an alkenyl group having 1-20 carbon atoms, an ether group having 1-20 carbon atoms, an alkoxy group having 1-20 carbon atoms, an aryl group having 6-30 carbon atoms, an alkaryl group having 6-30 carbon atoms, an aralkyl group having 6-30 carbon atoms, a cycloalkyl group having 3-10 carbon atoms, a substituted or an unsubstituted amine group, and a substituted sulfonate esterified group represented by the formula (1d):

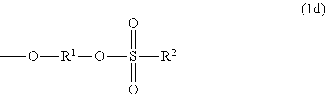

wherein R¹ is a substituent selected from a group comprising at least one aryl group comprising 6-30 carbon atoms, preferably 8-30 carbon atoms, preferably 8-20 carbon atoms, preferably 10-18 carbon atoms, preferably 11-18 carbon atoms; wherein R² is a substituent selected from a group consisting of a halogen substituted or an unsubstituted alkyl group having 1-20 carbon atoms, a branched or a linear alkyl group having 1-20 carbon atoms, a halogen substituted aryl group, a hydroxyl substituted aryl group, a nitro group substituted aryl group, an alkaryl group having 6-30 carbon atoms, an aralkyl group having 6-30 carbon atoms, a cycloalkyl group having 3-10 carbon atoms;
with the proviso that at least one of the substituent ‘A’, ‘B’, ‘E’, ‘Z’, ‘G’, ‘Q’ is a sulfonate esterified group as represented by the formula (1d). In other words, at least one of the phosphorous atom in the cyclic phosphazene compound of formula (1a) is substituted by the sulfonate esterified group as represented by the formula (1d).

Preferably in some embodiments of the disclosure each of the substituent ‘A’, ‘B’, ‘E’, ‘Z’, ‘G’, ‘Q’ is a sulfonate esterified group as represented by the formula (1d).

Preferably any alkyl group present as a substituent in the cyclic phosphazene compound of formula (1a) can have 1-20 carbon atoms, preferably 1-10 carbon atoms, preferably 1-5 carbon atoms. Preferably, the alkyl group is a methyl group, an ethyl group, a propyl group, a butyl group, an iso-propyl, an iso-butyl group, sec-butyl group, a pentyl group, a hexyl group.

Preferably the alkenyl group present as a substituent in the cyclic phosphazene compound of formula (1a) can have 1-20 carbon atoms, preferably 1-10 carbon atoms, preferably 1-5 carbon atoms. Preferably, the ether group can have 1-20 carbon atoms, preferably 1-10 carbon atoms, preferably 1-5 carbon atoms. Preferably, the alkoxy group present as a substituent in the cyclic phosphazene compound of formula (1a) can have 1-20 carbon atoms, preferably 1-10 carbon atoms, preferably 1-5 carbon atoms.

Preferably the aryl group present as a substituent in the cyclic phosphazene compound of formula (1a) can have 6-30 carbon atoms, preferably 6-18 carbon atoms, preferably 6-12 carbon atoms. Preferably, the alkaryl group present as a substituent in the cyclic phosphazene compound can have 6-30 carbon atoms, preferably 6-18 carbon atoms, preferably 6-12 carbon atoms. Preferably the aralkyl group present as a substituent in the cyclic phosphazene compound of formula (1a) can have 6-30 carbon atoms, preferably 6-18 carbon atoms, preferably 6-12 carbon atoms. Preferably the cycloalkyl group present as a substituent in the cyclic phosphazene compound of formula (1a) can have 3-10 carbon atoms, preferably 3-8 carbon atoms.

### Cyclic Phosphazene Compound

In some embodiments of the disclosure, referring to formula (1a), each of the variables ‘a’, ‘b’ and ‘c’ is 1. Preferably, the sulfonate esterified phosphazene compound is a cyclic phosphazene compound represented by formula (2):

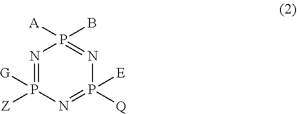

wherein each of the substituents ‘A’, ‘B’, ‘E’, ‘Z’, ‘G’, ‘Q’ are independently same or different, and are independently selected from a substituted or an unsubstituted alkyl group having 1-20 carbon atoms, a branched or a linear alkyl group having 1-20 carbon atoms, an alkenyl group having 1-20 carbon atoms, an aryl group having 6-30 carbon atoms, an ether group having 1-20 carbon atoms, an alkoxy group having 1-20 carbon atoms, an alkenyl substituted aryl group having 6-30 carbon atoms, an alkaryl group having 6-30 carbon atoms, an aralkyl group having 6-30 carbon atoms, a cycloalkyl group having 3-10 carbon atoms, a substituted or an unsubstituted amine group, and a substituted sulfonate esterified group represented by the formula (1d):

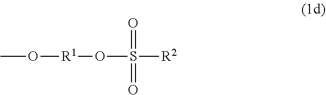

wherein R¹ is a substituent selected from a group comprising at least one aryl group comprising 6-30 carbon atoms, preferably 8-30 carbon atoms, preferably 8-20 carbon atoms, preferably 10-18 carbon atoms, preferably 11-18 carbon atoms;
wherein R² is a substituent selected from a group consisting of a halogen substituted or an unsubstituted alkyl group having 1-20 carbon atoms, a branched or a linear alkyl group having 1-20 carbon atoms, a halogen substituted aryl group, a hydroxyl substituted aryl group, a nitro group substituted aryl group, an alkaryl group having 6-30 carbon atoms, an aralkyl group having 6-30 carbon atoms, a cycloalkyl group having 3-10 carbon atoms;
with the proviso that at least one of the substituent ‘A’, ‘B’, ‘E’, ‘Z’, ‘G’, ‘Q’ is a sulfonate esterified group as represented by the formula (1d) as defined herein.

In other words, at least one of the phosphorous atom in the cyclic phosphazene formula (2) is substituted by the sulfonate esterified group as represented by the formula (1d).

Preferably, in some embodiments of the disclosure, each of ‘A’, ‘B’, ‘E’, ‘Z’, ‘G’, ‘Q’ is a sulfonate esterified group as represented by the formula (1d).

Preferably, each of ‘A’, ‘B’, ‘E’, ‘Z’, ‘G’, ‘Q’ is a sulfonate esterified group as represented by the formula (1d),

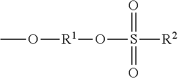

wherein the substituent R¹ is selected from the group consisting of:

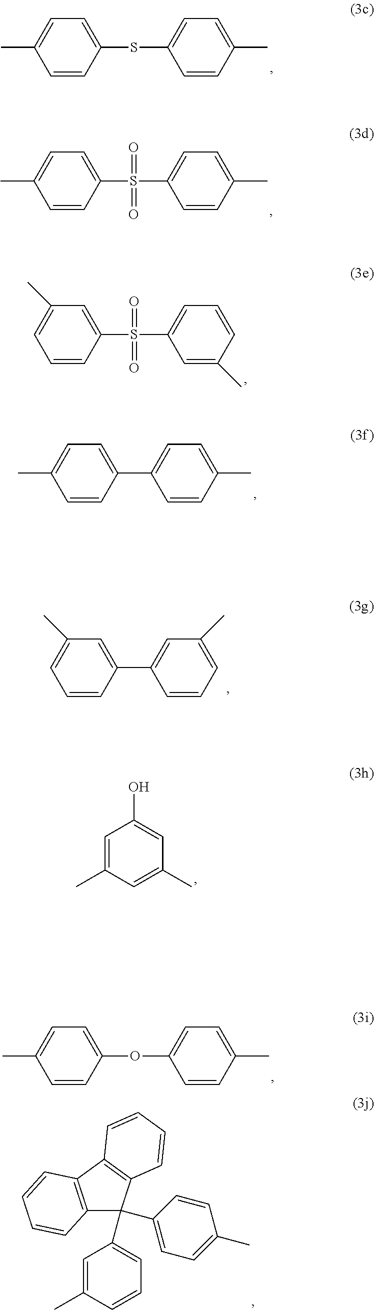

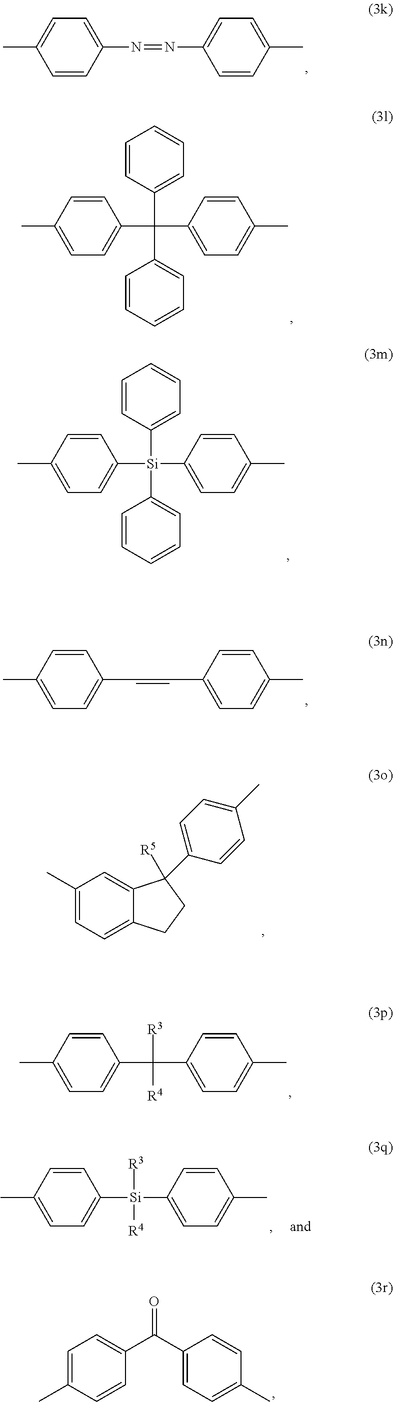

Preferably any alkyl group present as a substituent in the cyclic phosphazene compound of formula (2) can have 1-20 carbon atoms, preferably 1-10 carbon atoms, preferably 1-5 carbon atoms. Preferably, the alkyl group is a methyl group, an ethyl group, a propyl group, a butyl group, an iso-propyl, an iso-butyl group, sec-butyl group, a pentyl group, a hexyl group.

Preferably the alkenyl group present as a substituent in the cyclic phosphazene compound of formula (2) can have 1-20 carbon atoms, preferably 1-10 carbon atoms, preferably 1-5 carbon atoms. Preferably, the ether group can have 1-20 carbon atoms, preferably 1-10 carbon atoms, preferably 1-5 carbon atoms. Preferably, the alkoxy group present as a substituent in the cyclic phosphazene compound can have 1-20 carbon atoms, preferably 1-10 carbon atoms, preferably 1-5 carbon atoms.

Preferably the aryl group present as a substituent in the cyclic phosphazene compound of formula (2) can have 6-30 carbon atoms, preferably 6-18 carbon atoms, preferably 6-12 carbon atoms. Preferably, the alkaryl group present as a substituent in the cyclic phosphazene compound of formula (2) can have 6-30 carbon atoms, preferably 6-18 carbon atoms, preferably 6-12 carbon atoms. Preferably the aralkyl group present as a substituent in the cyclic phosphazene compound of formula (2) can have 6-30 carbon atoms, preferably 6-18 carbon atoms, preferably 6-12 carbon atoms. Preferably the cycloalkyl group of formula (2) present as a substituent in the cyclic phosphazene compound of formula (2) can have 3-10 carbon atoms, preferably 3-8 carbon atoms.

Referring to the substituted sulfonate esterified group represented by the formula (1d):

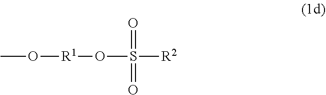

the substituent R¹ is selected from the group consisting of:

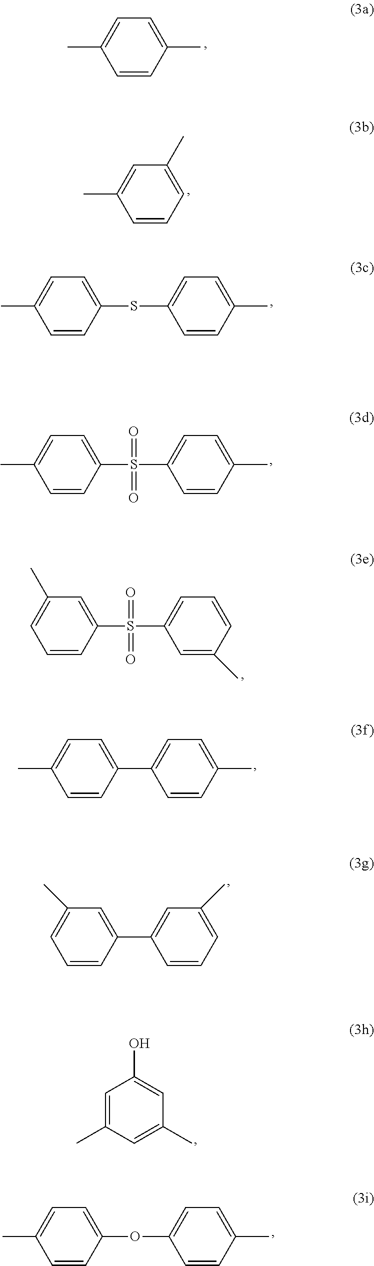

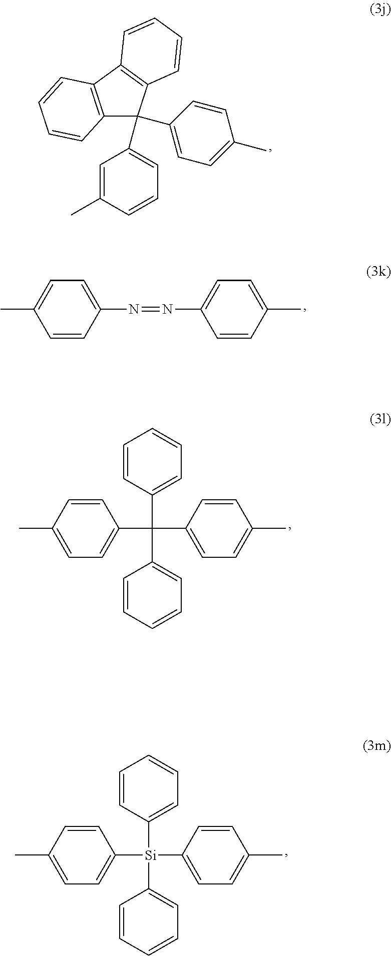

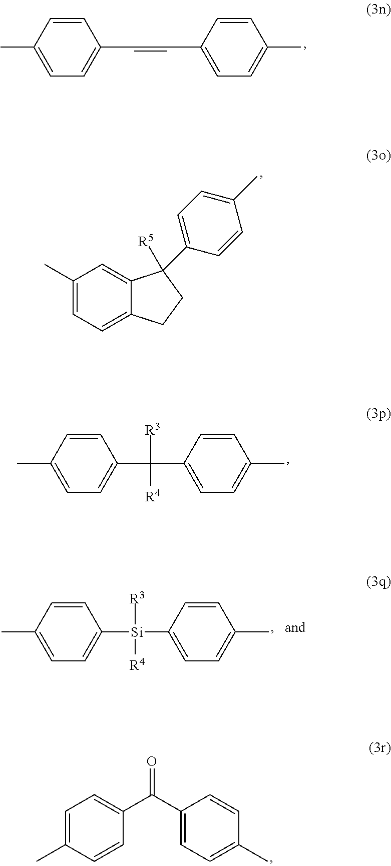

wherein the substituents R³, R⁴, R⁵, are each independently selected from the group consisting of a halogen substituted or an unsubstituted alkyl group having 1-10 carbon atoms, and a branched or a linear alkyl group having 1-10 carbon atoms.

Preferably, any alkyl group present as a substituent can have 1-20 carbon atoms, preferably 1-10 carbon atoms, preferably 1-5 carbon atoms. Preferably, the alkyl group is a methyl group, an ethyl group, a propyl group, a butyl group, an iso-propyl, an iso-butyl group, sec-butyl group, a pentyl group, a hexyl group. Preferably, the halogen group is fluorine group. For each of the formulas and structures provided above, the two points of attachments for the substituent R¹ are indicated by the projected nodes denoted in each of the structures or formulas.

Preferably, R¹ is a substituent selected from a group comprising at least one aryl group comprising 8-30 carbon atoms, preferably 8-20 carbon atoms, preferably 10-18 carbon atoms, preferably 11-18 carbon atoms. Preferably, the substituted sulfonate esterified group represented by the formula (1d):

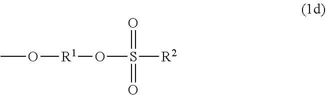

the substituent R¹ is selected from the group consisting of:

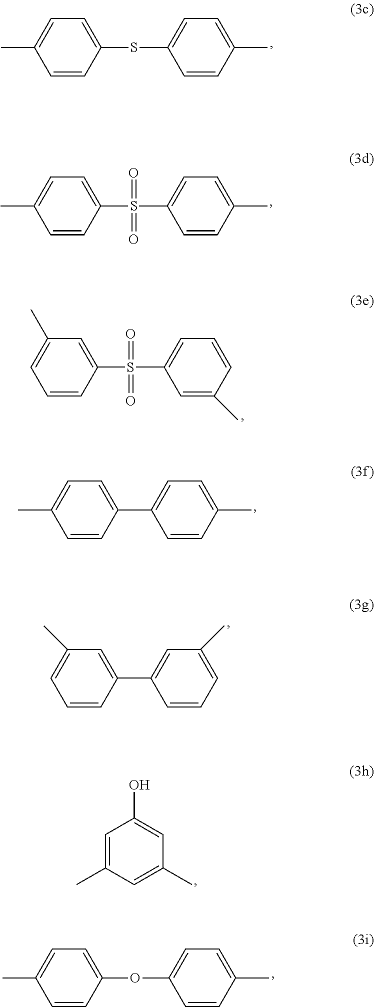

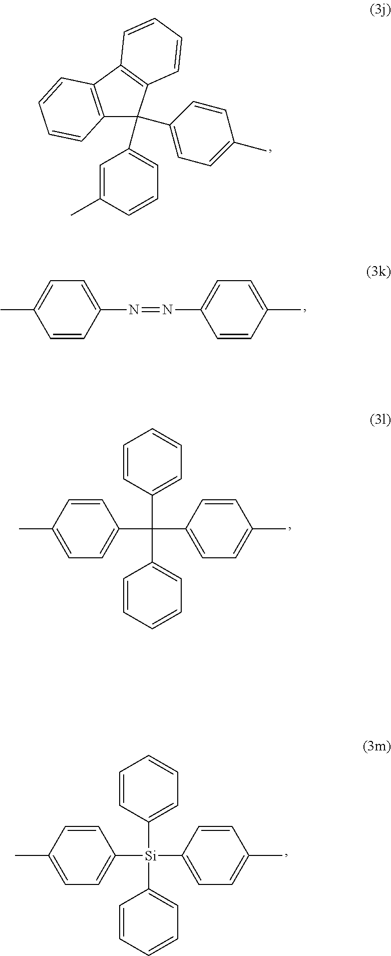

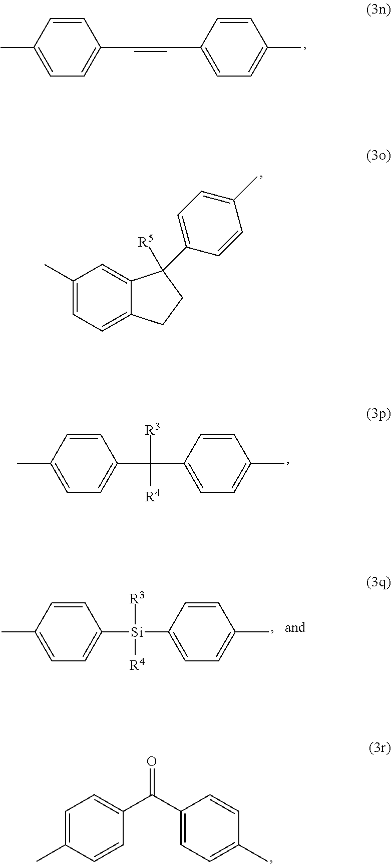

wherein the substituents R³, R⁴, R⁵, are each independently selected from the group consisting of a halogen substituted or an unsubstituted alkyl group having 1-10 carbon atoms, and a branched or a linear alkyl group having 1-10 carbon atoms.

Preferably, any alkyl group present as a substituent can have 1-20 carbon atoms, preferably 1-10 carbon atoms, preferably 1-5 carbon atoms. Preferably, the alkyl group is a methyl group, an ethyl group, a propyl group, a butyl group, an iso-propyl, an iso-butyl group, sec-butyl group, a pentyl group, a hexyl group. Preferably, the halogen group is fluorine group. For each of the formulas and structures provided above, the two points of attachments for the substituent R¹ are indicated by the projected nodes denoted in each of the structures or formulas

Preferably, each of the substituents R³, R⁴, R⁵ is a methyl or an ethyl group, preferably each of the substituents R³, R⁴, R⁵ is a methyl group (—CH₃). Preferably each of the substituents R³, R⁴, R⁵ is a fluorine substituted alkyl group. More preferably each of the substituents R³, R⁴, R⁵ is a fluorine substituted alkyl group represented by the formula (—CF₃).

Referring to the substituted sulfonate esterified group represented by the formula (1d):

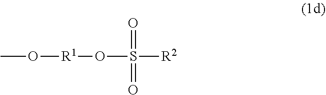

the substituent R² is selected from the group consisting of:

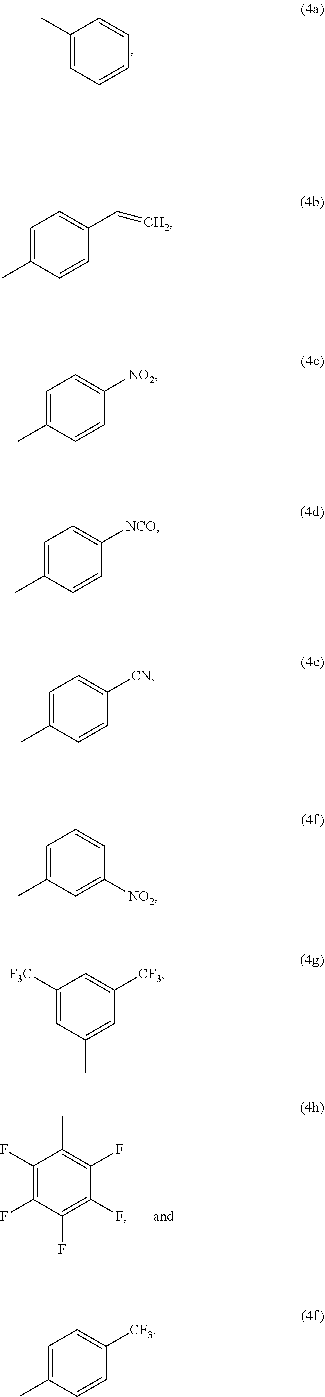

For each of the formulas and structures provided above, the single point of attachments for the substituent R² is indicated by the projected nodes denoted in each of the structures or formulas.

In some preferred embodiments of the disclosure, the sulfonate esterified phosphazene compound is a cyclic phosphazene compound represented by the formula (5):

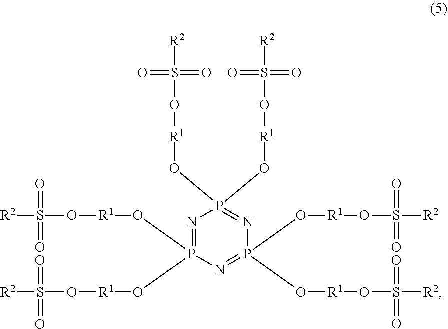

wherein the substituents R¹ and
R² are as defined herein.

Preferably, R¹ is a substituent selected from a group comprising at least one aryl group comprising 6-30 carbon atoms, preferably 8-30 carbon atoms, preferably 8-20 carbon atoms, preferably 10-18 carbon atoms, preferably 11-18 carbon atoms.

Preferably, in some embodiments of the disclosure, the sulfonate esterified phosphazene compound is a cyclic phosphazene compound represented by the formula:

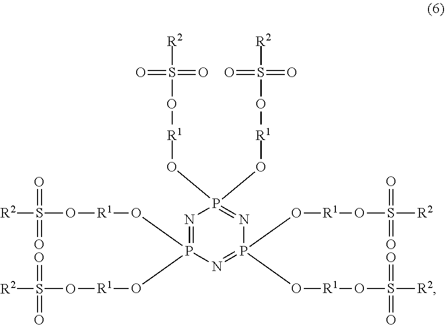

wherein each of the substituent R¹ is

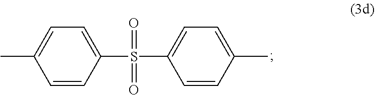

and the substituent R² is

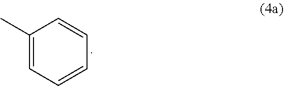

Preferably, in some embodiments of the disclosure, the sulfonate esterified phosphazene compound is a cyclic phosphazene compound represented by the formula:

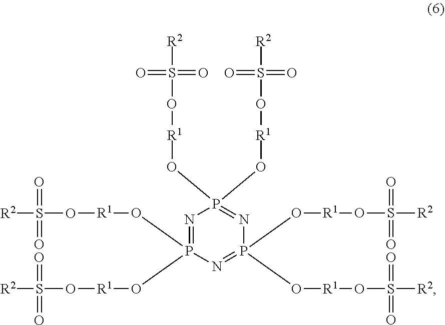

wherein each of the substituent R¹ is

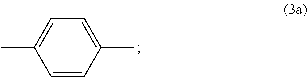

and the substituent R² is

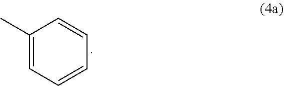

Preferably, in some embodiments of the disclosure, the sulfonate esterified phosphazene compound is a cyclic phosphazene compound represented by the formula:

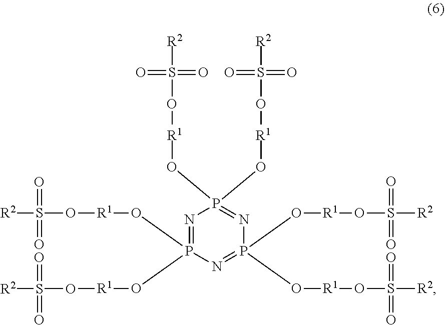

wherein each of the substituent R¹ is

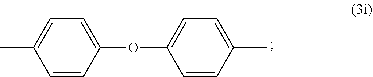

and the substituent R² is

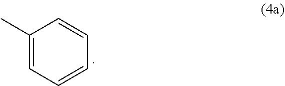

In some preferred embodiments of the disclosure, the sulfonate esterified phosphazene compound is hexa (benzenesulfonic acid 4-(4-oxy-benzenesulfonyl)-phenyl ester) cyclotriphosphazene (HSSCP) represented by the formula:

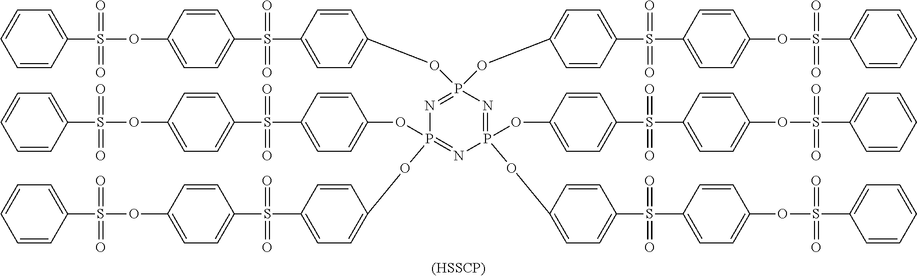

In some embodiments of the disclosure, the disclosure is directed to a method of preparing a sulfonate esterified phosphazene compound, comprising the steps of:
i. reacting in presence of a base, a diol compound (V) represented by the formula

HO—R¹—OH
with a sulfonyl halide compound represented by the formula (W)

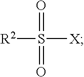

and forming a first intermediate compound; wherein the substituents R¹ and R² is as defined herein in this disclosure; wherein the substituent ‘X’ is a halogen; preferably wherein the substituent ‘X’ is chlorine; and
ii. reacting in presence of a base, the first intermediate compound with a phosphazene compound (T) having at least one phosphorous atom substituted by a nucleophilic leaving group (NLG), and forming a sulfonate esterified phosphazene compound;
   wherein the nucleophilic leaving group (NLG) is selected from the group consisting of a halogen group, a thio group, a cyano group, a tosylate group, an alkoxy group, an aryloxy group, an alkyl ester group, an azide group, an amine group, preferably the nucleophilic leaving group (NLG), is a halogen group. More preferably, the halogen group is chlorine.

Alternatively, in some embodiments of the disclosure, the disclosure is directed to a method of preparing a sulfonate esterified phosphazene compound, comprising the steps of:
i. reacting in presence of a base, a diol compound (V) represented by the formula

HO—R¹—OH

with a phosphazene compound (T) having at least one phosphorous atom substituted by a nucleophilic leaving group (NLG) and forming a second intermediate compound; and
ii. reacting in presence of a base, the second intermediate compound with a sulfonyl halide compound represented by the formula (W)

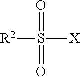

and forming a sulfonate esterified phosphazene compound;
wherein the substituents R¹ and R² is as defined throughout this disclosure; wherein the substituent ‘X’ is a halogen; preferably wherein the substituent ‘X’ is chlorine;
wherein the nucleophilic leaving group (NLG) is selected from the group consisting of a halogen group, a thio group, a cyano group, a tosylate group, an alkoxy group, an aryloxy group, an alkyl ester group, an azide group, an amine group, preferably the nucleophilic leaving group (NLG), is a halogen group. More preferably, the halogen group is chlorine.

Preferably, the base is selected from KOH, NaOH, LiOH, NaH, KH, K₂CO₃, Na₂CO₃, LizCO₃, K₃PO₄, Pyridine, 4-Dimethylaminopyridine, trimethylamine, triethylamine or any combinations thereof. Preferably, the base used in step (i) and step (ii) of the method of preparing the sulfonate esterified phosphazene compound is identical. Preferably, the base used in step (i) and step (ii) of the method of preparing the sulfonate esterified phosphazene compound is not identical. The expression “nucleophilic leaving group (NLG)” as used throughout this disclosure, is a functional group or an atom capable of being displaced in a nucleophilic substitution reaction.

Preferably, the phosphazene compound (T) is a cyclic phosphazene compound represented by the structure:

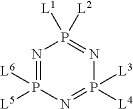

wherein the substituents L¹, L², L³, L⁴, L⁵ and L⁶ are independently same or different, and are independently selected from the nucleophilic leaving group (NLG), a substituted or an unsubstituted alkyl group having 1-20 carbon atoms, a branched or a linear alkyl group having 1-20 carbon atoms, an alkenyl group having 1-20 carbon atoms, an aryl group having 6-30 carbon atoms, an alkaryl group having 6-30 carbon atoms, an alkenyl substituted aryl group having 6-30 carbon atoms, an aralkyl group having 6-30 carbon atoms, a cycloalkyl group having 3-10 carbon atoms, a substituted or an unsubstituted amine group, with the proviso that at least one of the substituent L¹, L², L³, L⁴, L⁵ or L⁶ is a nucleophilic leaving group (NLG), preferably all the substituents L¹, L², L³, L⁴, L⁵ and L⁶ are a nucleophilic leaving group (NLG), preferably each of the substituents L¹, L², L³, L⁴, L⁵ and L⁶ is an halogen, preferably each of the substituents L¹, L², L³, L⁴, L⁵ and L⁶ is chlorine. Preferably, the phosphazene compound (T) is hexa-chloro-cyclotriphosphazene (HCC). Preferably, the diol compound (V) is bis(4-hydroxyphenyl) sulfone. Preferably, the sulfonyl halide compound (W) is benzenesulfonyl chloride.

Preferably, in some embodiments of the disclosure, the phosphazene compound (T) is a cyclic phosphazene compound represented by the formula:

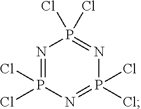 [SMILES: ClP1(Cl)=NP(Cl)(Cl)=NP(Cl)(Cl)=N1]

the diol compound (V) is represented by the formula:

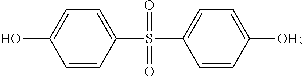 [SMILES: O=S(=O)(c1ccc(O)cc1)c1ccc(O)cc1]

and the sulfonyl halide compound (W) is represented by the formula:

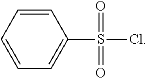

In some embodiments of the disclosure, the disclosure is directed to a polymer composition comprising:
i. ≥80.0 wt. % and ≤99.95 wt. %, preferably ≥85.0 wt. % and ≤99.95 wt. %, with regard to the total weight of the composition, of a thermoplastic polymer; and
ii. ≥0.05 wt. % and ≤20.0 wt. %, preferably ≥0.05 wt. % and ≤15.0 wt. %, preferably ≥1.0 wt. % and ≤5.0 wt. %, with regard to the total weight of the composition, of the sulfonate esterified phosphazene compound of the present disclosure.

Preferably, in some embodiments of the disclosure, the disclosure is directed to a polymer composition comprising:
i. ≥75.0 wt. % and ≤99.95 wt. %, ≥80.0 wt. % and ≤99.95 wt. %, preferably ≥85.0 wt. % and ≤99.95 wt. %, with regard to the total weight of the composition, of a thermoplastic polymer;
ii. ≥0.05 wt. % and ≤20.0 wt. %, preferably ≥0.05 wt. % and ≤15.0 wt. %, preferably ≥1.0 wt. % and ≤5.0 wt. %, with regard to the total weight of the composition, of the sulfonate esterified phosphazene compound of the present disclosure; and
iii. optionally, ≥0.0 wt. % and ≤5.0 wt. %, preferably ≥0.0 wt. % and ≤1.5 wt. %, preferably ≥0.4 wt. % and ≤1.0 wt. % of with regard to the total weight of the composition, of additives.

Non-liming examples of additives may be anti-oxidant, processing aid, color pigment, light stabilizers, diffuser particles. Preferably in some embodiments of the disclosure, the thermoplastic polymer is selected from polyethylene terephthalate (PET), impact polypropylene copolymers, propylene ethylene copolymers, propylene ethylene alpha olefin terpolymers, polycarbonate (PC), polybutylene terephthalate (PBT), poly(1,4-cyclohexylidene cyclohexane-1,4-dicarboxylate) (PCCD), glycol modified polycyclohexyl terephthalate (PCTG), poly(phenylene oxide) (PPO), polypropylene (PP), polyethylene (PE), polyvinyl chloride (PVC), ethylene-C₄-C₁₂-alpha-olefin copolymers, polystyrene (PS), polymethyl methacrylate (PMMA), polyethyleneimine or polyetherimide (PEI) and their derivatives, thermoplastic elastomer (TPE), terephthalic acid (TPA) elastomers, poly(cyclohexanedimethylene terephthalate) (PCT), polyethylene naphthalate (PEN), polyamide (PA), polysulfone sulfonate (PSS), sulfonates of polysulfones, polyether ether ketone (PEEK), acrylonitrile butyldiene styrene (ABS), polyether ketone ketone (PEKK), polyphenylene sulfide (PPS), co-polymers thereof, or blends thereof.

Preferably, the thermoplastic polymer is selected from polycarbonate, polyethylene, and polypropylene. Preferably, the thermoplastic polymer is a polycarbonate. The polycarbonate may be selected from a linear polycarbonate, a branched polycarbonate, a polycarbonate-siloxane copolymer, and combinations thereof.

Preferably wherein the polycarbonate is a linear polycarbonate comprising repeat units derived from bisphenol A carbonate. Preferably, the polycarbonate is a combination of a linear polycarbonate comprising repeat units derived from bisphenol A carbonate and polycarbonate-siloxane copolymer.

In some embodiments of the disclosure, the disclosure is directed to an article comprising the polymeric composition. The article comprising the polymer composition may be prepared by the steps involving: (i) dry blending the thermoplastic polymer and the sulfonate esterified phosphazene compound of the present disclosure; and (ii) extruding the blend in an extruder at a melt temperature <240° C. and forming the article.

In some embodiments of the disclosure, the disclosure is directed to the use of the sulfonate esterified phosphazene compound for improving fire retardant properties of a polymer composition.

Specific examples demonstrating some of the embodiments of the disclosure are included below. The examples are for illustrative purposes only and are not intended to limit the disclosure. It should be understood that the embodiments and the aspects disclosed herein are not mutually exclusive and such aspects and embodiments can be combined in any way. Those of ordinary skill in the art will readily recognize parameters that can be changed or modified to yield essentially the same results.

### Example I

Purpose: To synthesize a cyclic a sulfonate esterified phosphazene compound hexa (benzenesulfonic acid 4-(4-oxy-benzenesulfonyl)-phenyl ester) cyclotriphosphazene (HSSCP) also referred to as Compound (II) for the purpose of this example section:

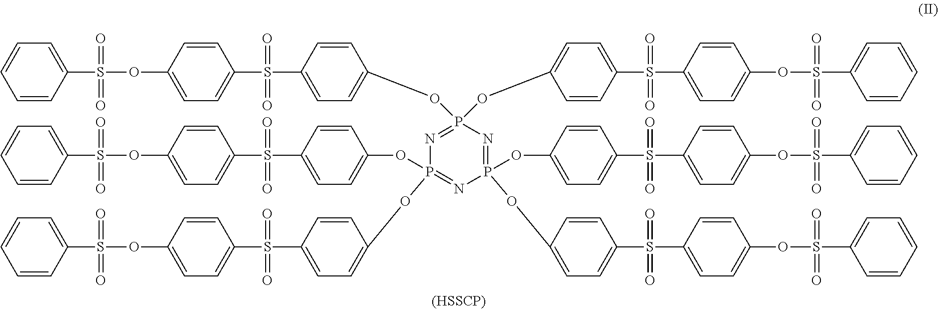

Material Details: The following materials were used: Bis(4-hydroxyphenyl) sulfone (98%, Sigma-Aldrich), Benzenesulfonyl chloride (BSC) (99%, Sigma-Aldrich), Hexachlorocyclotriphosphazene (HCCP, 99%, Sigma-Aldrich), anhydrous trimethylamine (Sigma-Aldrich) and solvents were used without further purification. Fourier transform infrared spectra (FT-IR) were acquired from KBr pellets of samples using a NICOLET-6700 FT-IR spectrometer. NMR spectra were recorded with a Bruker AVANCE-III 500 MHz spectrometer in deuterated chloroform. Carbon, nitrogen, sulfur and hydrogen contents of the samples were determined by a Flash 2000 CHNS/O organic elemental analyzer (Thermo Fisher Scientific).

Overall reaction Scheme for the synthesis: The following reaction scheme was used for the synthesis:

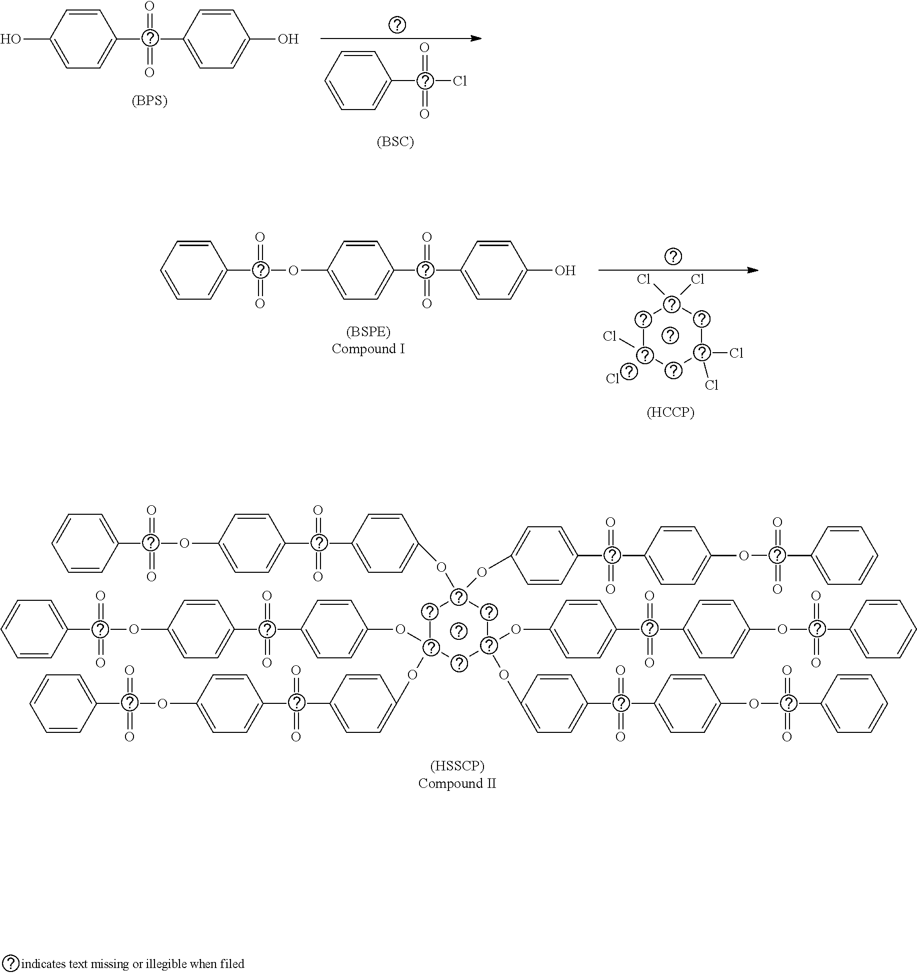

Preparation of Benzenesulfonic acid 4-(4-hydroxy-benzenesulfonyl)-phenyl ester (BSPE) (Compound I) as an intermediate: To a solution of bis(4-hydroxyphenyl) sulfone (20 g, 80 mmol) in anhydrous acetonitrile (300 ml) was added Et₃N (16.7 ml, 120 mmol). Then, benzenesulfonyl chloride (BSC) (80 mmol, 10.2 ml of BSC in 50 ml of anhydrous acetonitrile) was added dropwise. The reaction was refluxed overnight. The reaction solution was concentrated and precipitated in water. A white solid was obtained. The compound (I) was purified by silica gel column using ethyl acetate/hexane=½.

Spectral analysis of Compound I: ¹H-NMR (CDCl3, δ, ppm): δ=7.85 (m, 4H, ArH), 7.79 (m, 2H, ArH), 7.72 (m, 1H, ArH), 7.57 (m, 2H, ArH); 7.14 (m, 2H, ArH); 6.93 (m, 2H, ArH); ¹³C-NMR (CDCl3, δ, ppm): δ=160.41, 152.53, 141.03, 134.89, 134.75, 132.32, 130.26, 129.44, 129.24, 128.41, 123.17, 116.25; FT-IR (KBr, cm⁻¹): v 3408 s (OH), 3099 m ((C—H) arom.), 1589, 1490 s (C═C), 1377 s (O═S═O), 1145 s (C—O), 1008 s (S—O) Anal. Calculated for C₁₈H₁₄O₆S₂: C, 55.37; H, 3.61; S, 16.43. Found C, 55.78; H, 3.58; S, 17.72.

Preparation of hexa (benzenesulfonic acid 4-(4-oxy-benzenesulfonyl)-phenyl ester) cyclotriphosphazene (Compound II): A solution of Benzenesulfonic acid 4-(4-hydroxy-benzenesulfonyl)-phenyl ester or compound (I) (7.77 g, 19.9 mmol in 200 ml of anhydrous acetonitrile) was added to 4 ml of triethylamine (28.7 mmol). A solution of hexachlorophosphazene (HCCP) (1.05 g (3 mmol) in 50 ml anhydrous acetonitrile) was added dropwise to the above reaction mixture and continued stirring at r.t. and then reflux overnight. The reaction solution was concentrated and precipitated in water. A white solid was obtained. The compound (II) was purified by silica gel column using ethyl acetate/hexane=1/1.

Spectral analysis of Compound II: ¹H-NMR (CDCl3, δ, ppm): δ=7.97 (m, 12H, ArH), 7.86 (m, 24H, ArH), 7.72 (m, 6H, ArH), 7.58 (m, 12H, ArH); 7.21 (m, 12H, ArH); 6.88 (m, 12H, ArH); ¹³C-NMR (CDCl3, δ, ppm): δ=153.34, 153.07, 139.50, 138.95, 134.91, 134.83, 130.00, 129.97, 129.50, 128.38; ³¹P-NMR (CDCl3, δ, ppm): δ=7.52 (s, IP, cyclotriphosphazene). FTIR (KBr, cm⁻¹): v 3099 m ((C—H) arom.), 1587, 1487 s (C═C), 1380 s (O═S═O), 1293 (P═N), 1153 (C—O), 1014 (S—O), 949 (P—O), MS: m/z=2514.02 [M-H]⁺+formic acid adduct. Anal. Calculated for C₁₀₈H⁷⁸N₃O₃₆P₃S₁₂: C, 52.49; H, 3.81; N, 1.70; S, 15.57. Found C, 52.62; H, 3.07; N, 1.95; S, 15.37.

The spectral analysis evidenced the synthesis of Compound II using the intermediate product Compound I. The spectral analysis also showed all the phosphorus atoms were substituted by the sulfonate esterified groups.

### Example II

Purpose: Evaluate the flame retardant properties of Compound II in polycarbonate compositions. For the purpose of the present examples, two different polycarbonate (PC) compositions were formulated and their flame retardant properties were evaluated using the standard UL94 vertical burning test protocols and the related UL rating judgement criteria.

Materials: The following polycarbonate formulations were prepared and the formulations details are presented in the table below:

TABLE 1

| | | | Sample 2, Wt % |
|---|---|---|---|
| | | Sample 1, Wt % | (Compound |
| Component | Description | (Compound II/PC) | II/PC/PC-Si) |
| PC 1 | BPA polycarbonate (Mw = | 75.00 | 37.65 |
| | 30,500 g/mol) | | |
| PC 2 | BPA polycarbonate (Mw = | 21.09 | 42.00 |
| | 21,800 g/mol) | | |
| PC-Si | BPA-siloxane copolycarbonate | 0 | 17.50 |
| | (20% siloxane, Mw = 30,000 | | |
| | g/mol) | | |
| Compound II | Hexa(benzenesulfonic acid 4- | 3.00 | 2.00 |
| | (4-oxy-benzenesulfonyl)- | | |
| | phenyl ester) | | |
| | cyclotriphosphazene | | |
| PETS | Pentaerythritol tetrastearate | 0.35 | 0.30 |
| Irgafos 168 | Tris(2,4-di-tert- | 0.06 | 0.05 |
| | butylphenyl)phosphite | | |
| TSAN | Styrene-acrylonitrile (SAN)- | 0.50 | 0.50 |
| | encapsulated PTFE | | |

Process for preparation of samples: The samples were prepared by melt blending. All powder additives were combined together with the polycarbonate (PC) powder, using a paint shaker, and fed through a feeder to an extruder (ZSK25, Co-Rotating, Twin Screw Extruder).

Flame Test Standard: The samples obtained were evaluated for their flame retardant property. The vertical burning tests for UL94 combustion level were measured on an FTT 0082 (Fire Testing Technology, UK) instrument with the sample dimensions of 130 mm×13 mm×1.2 mm according to the ASTM D3801 testing procedure. In the test, five sample bars suspended vertically over surgical cotton were ignited using a 50 W methane gas burner. The flammability properties of all investigated formulations were measured following the standard UL94 vertical burning test protocols and the related UL rating judgement criteria are listed in Table 2.

The flammability properties of all investigated formulations were determined by UL94 vertical flammability tests at 1.2 mm thicknesses for five flame bars for each sample. The degree of flame retardant may be evaluated based on the rating given to a material with the extent of flame retardant properties indicated by V0>V1>V2 with V0 having the best flame retardant rating and V2 being the least.

TABLE 2

| | | | | N.R. |
|---|---|---|---|---|
| | | | | (No |
| Rating criteria | V-0 | V-1 | V-2 | rating) |
| After flame time for each individual | <10 s | <30 s | <30 s | >30 s |
| specimen t1 or t2 | | | | |
| Total flame-out-time for all 5 bars | <50 s | <250 s | <250 s | >250 s |
| (FOT = t1 + t2) | | | | |
| After flame or after glow of any | No | No | No | |
| specimen up to the holding clamp | | | | |
| Cotton indicator ignited by flaming | No | No | Yes | |
| particles or drops | | | | |

Result and Conclusion: The results from the flame retardant test are reported under Table 3. As is evident from the UL-94 rating data, the addition of the compound hexa (benzenesulfonic acid 4-(4-oxy-benzenesulfonyl)-phenyl ester) cyclotriphosphazene (Compound II) in the polycarbonate compositions (Sample 1 and 2), resulted in the improved flame retardant property compared to the flame retardant property of comparative sample (CE) which did not contain Compound II. For example, the comparative sample (CE) comprising BPA polycarbonate resin without the Compound II, has a flame retardant rating of UL-94 V-2 while samples Sample I and Sample 2 have better UL-94 ratings.

In particular, Sample 1 containing 3.0% of Compound II complied with the rating of UL94 V-1 while Sample 2 containing 2.0% of Compound II complied with the rating requirements of UL94 V0.

Further, the Compound II was subjected to thermal degradation test. It was found that the Compound II (HSSCP) demonstrated suitable thermal stability. It was observed during testing that the onset of the decomposition temperature, (T5%, set to be when the mass loss equaled 5 wt. %), was found to be T5%=402.9° C. and 399.08° C. for HSSCP under N₂ and air. Accordingly, Compound II can be used effectively as flame retardant in polymer compositions.

TABLE 3

| | | | | | Cot- | |
|---|---|---|---|---|---|---|
| | Loading of | t1 | 5-bar | | ton | |
| | Compound II | average | FOT | Drip- | igni- | |
| Samples | (Wt %) | (s) | (s) | pings | tion | UL-94 |
| Inventive | 3.0 | 11 | 65 | No | No | V1 |
| (Sample I) | | | | | | |
| Inventive | 2.0 | 4 | 15 | No | No | V0 |
| (Sample 2) | | | | | | |
| Comparative | 0.0 | <30 s | <250 s | No | Yes | V2 |
| Sample (CE) | | | | | | |
| (BPA | | | | | | |
| polycarbonate | | | | | | |
| (Mw = 30,500 | | | | | | |
| g/mol) | | | | | | |

1. A sulfonate esterified phosphazene compound selected from the group consisting of:
i. a cyclic phosphazene compound represented by the formula 1a: 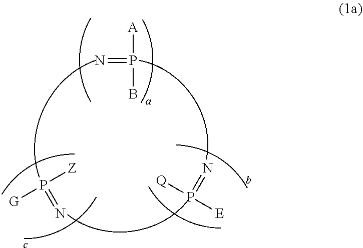
wherein each of the variables ‘a’, ‘b’ and ‘c’ are integers ranging from ≥1 and ≤5, or each of the variables ‘a’, ‘b’ and ‘c’ is 1;
ii. a linear phosphazene compound represented by the formula (1b): 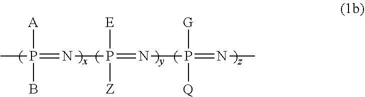
wherein each of the variables ‘x’, ‘y’ and ‘z’ are integers ranging from ≥1 and ≤500; from ≥50 and ≤200, or from ≥100 and ≤200; and
iii. a cross-linked phosphazene compound represented by the formula (1c): 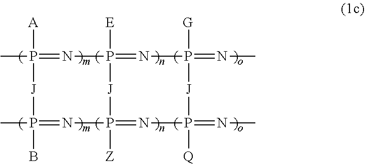
wherein each of the variables ‘m’, ‘n’ and ‘o’ are integers ranging from ≥1 and ≤500, from ≥50 and ≤200, or from ≥100 and ≤200;
wherein each of the substituents ‘A’, ‘B’, ‘E’, ‘Z’, ‘G’, ‘Q’, ‘J’ are independently the same or different, wherein each of the substituents ‘A’, ‘B’, ‘E’, ‘Z’, ‘G’, ‘Q’, ‘J’ are independently selected from a substituted or an unsubstituted alkyl group having 1-20 carbon atoms, a branched or a linear alkyl group having 1-20 carbon atoms, an alkenyl group having 1-20 carbon atoms, an ether group having 1-20 carbon atoms, an alkoxy group having 1-20 carbon atoms, an aryl group having 6-30 carbon atoms, an alkaryl group having 6-30 carbon atoms, an aralkyl group having 6-30 carbon atoms, a cycloalkyl group having 3-10 carbon atoms, a substituted or an unsubstituted amine group, and a substituted sulfonate esterified group represented by the formula (1d): 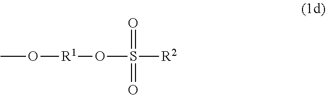
wherein R¹ is a substituent selected from a group comprising at least one aryl group comprising 6-30 carbon atoms, 8-30 carbon atoms, 8-20 carbon atoms, 10-18 carbon atoms, or 11-18 carbon atoms;
wherein R² is a substituent selected from a group consisting of a halogen substituted or an unsubstituted alkyl group having 1-20 carbon atoms, a branched or a linear alkyl group having 1-20 carbon atoms, a halogen substituted aryl group, a hydroxyl substituted aryl group, a nitro group substituted aryl group, an alkaryl group having 6-30 carbon atoms, an aralkyl group having 6-30 carbon atoms, and a cycloalkyl group having 3-10 carbon atoms;
with the proviso that at least one of the substituents ‘A’, ‘B’, ‘E’, ‘Z’, ‘G’, ‘Q’ is a sulfonate esterified group as represented by the formula (1d), or wherein each of the substituents ‘A’, ‘B’, ‘E’, ‘Z’, ‘G’, ‘Q’ is a sulfonate esterified group as represented by the formula (1d).

2. The sulfonate esterified phosphazene compound of claim 1, wherein the sulfonate esterified phosphazene compound is a cyclic phosphazene compound represented by formula (2): 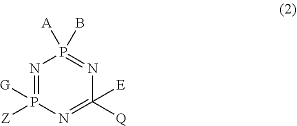
wherein each of the substituents ‘A’, ‘B’, ‘E’, ‘Z’, ‘G’, ‘Q’ are independently the same or different, and are independently selected from a substituted or an unsubstituted alkyl group having 1-20 carbon atoms, a branched or a linear alkyl group having 1-20 carbon atoms, an alkenyl group having 1-20 carbon atoms, an aryl group having 6-30 carbon atoms, an ether group having 1-20 carbon atoms, an alkoxy group having 1-20 carbon atoms, an alkenyl substituted aryl group having 6-30 carbon atoms, an alkaryl group having 6-30 carbon atoms, an aralkyl group having 6-30 carbon atoms, a cycloalkyl group having 3-10 carbon atoms, a substituted or an unsubstituted amine group, and a substituted sulfonate esterified group represented by the formula (1d): 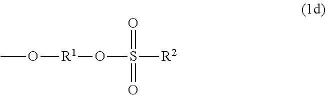
wherein R¹ is a substituent selected from a group comprising at least one aryl group comprising 6-30 carbon atoms, 8-30 carbon atoms, 8-20 carbon atoms, 10-18 carbon atoms, or 11-18 carbon atoms;
wherein R² is a substituent selected from a group consisting of a halogen substituted or an unsubstituted alkyl group having 1-20 carbon atoms, a branched or a linear alkyl group having 1-20 carbon atoms, halogen substituted aryl group, a hydroxyl substituted aryl group, a nitro group substituted aryl group, an alkaryl group having 6-30 carbon atoms, an aralkyl group having 6-30 carbon atoms, and a cycloalkyl group having 3-10 carbon atoms;
with the proviso that at least one of the substituents ‘A’, ‘B’, ‘E’, ‘Z’, ‘G’, ‘Q’ is a sulfonate esterified group as represented by the formula (1d) as defined in claim 1, or wherein each of the substituents of ‘A’, ‘B’, ‘E’, ‘Z’, ‘G’, ‘Q’ is a sulfonate esterified group as represented by the formula (1d).

3. The sulfonate esterified phosphazene compound claim 1, wherein the substituent R¹ is selected from the group consisting of: 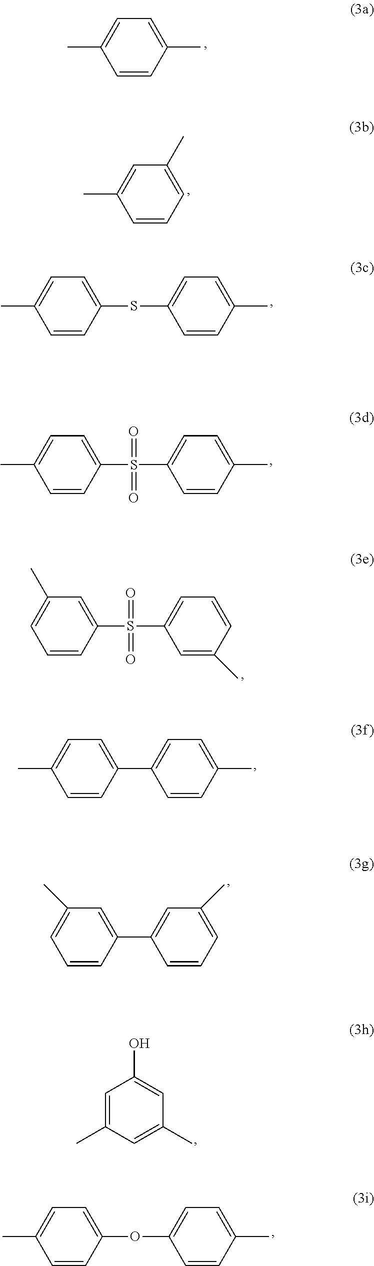 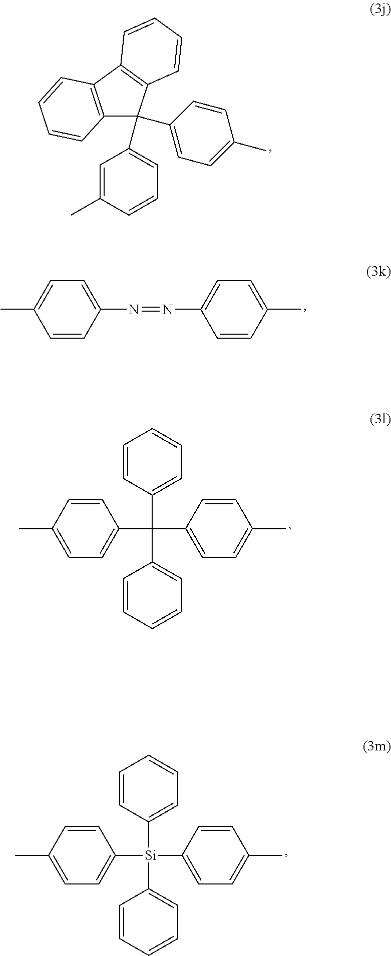 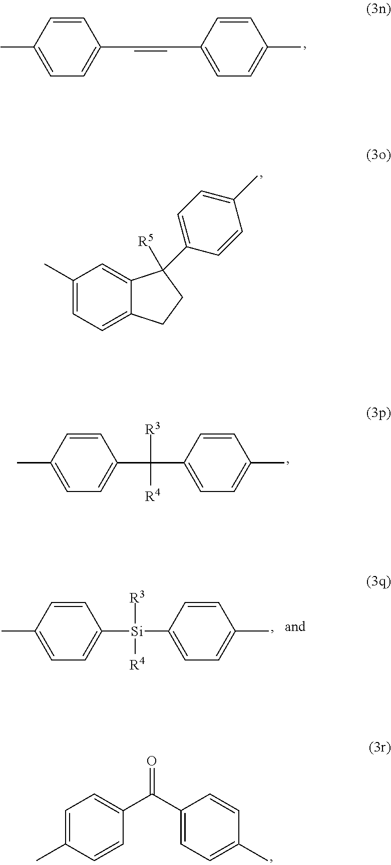
wherein the substituents R³, R⁴, and R⁵ are each independently selected from the group consisting of a halogen substituted or an unsubstituted alkyl group having 1-10 carbon atoms, and a branched or a linear alkyl group having 1-10 carbon atoms.

4. The sulfonate esterified phosphazene compound claim 1, wherein the substituent R² is selected from the group consisting of:

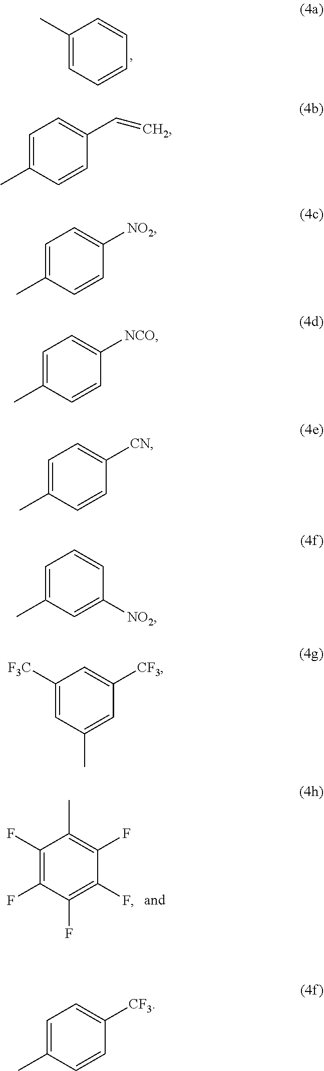

5. The sulfonate esterified phosphazene compound claim 1, wherein the sulfonate esterified phosphazene compound is a cyclic phosphazene compound represented by the formula:

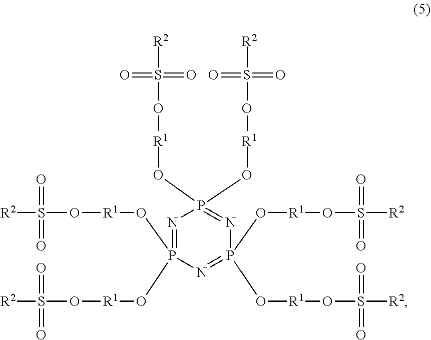

6. The sulfonate esterified phosphazene compound claim 1, wherein the sulfonate esterified phosphazene compound is a cyclic phosphazene compound represented by the formula: 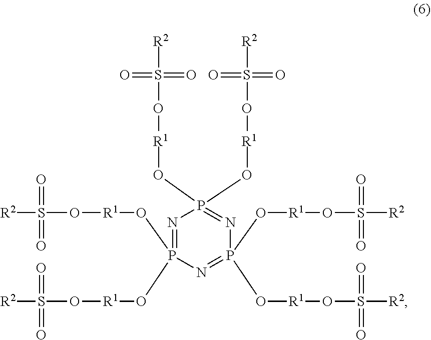
wherein each of the substituent R¹ is 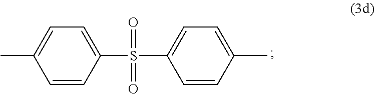
and the substituent R² is

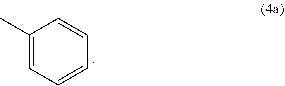

7. A method of preparing a sulfonate esterified phosphazene compound, comprising the steps of:
i. reacting in the presence of a base, a diol compound (V) represented by the formula
HO—R¹—OH
with a sulfonyl halide compound represented by the formula (W) 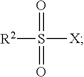
and forming a first intermediate compound; wherein the substituents R¹ and R² are as defined in claim 1; wherein the substituent ‘X’ is a halogen; and
ii. reacting in the presence of a base, the first intermediate compound with a phosphazene compound (T) having at least one phosphorous atom substituted by a nucleophilic leaving group (NLG), and forming a sulfonate esterified phosphazene compound;
wherein the nucleophilic leaving group (NLG) is selected from the group consisting of a halogen group, a thio group, a cyano group, a tosylate group, an alkoxy group, an aryloxy group, an alkyl ester group, an azide group, and an amine group.

8. A method of preparing a sulfonate esterified phosphazene compound, comprising the steps of:
i. reacting in the presence of a base, a diol compound (V) represented by the formula
HO—R¹—OH
with a phosphazene compound (T) having at least one phosphorous atom substituted by a nucleophilic leaving group (NLG) and forming a second intermediate compound; and
ii. reacting in the presence of a base, the second intermediate compound with a sulfonyl halide compound represented by the formula (W) 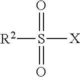
and forming a sulfonate esterified phosphazene compound;
wherein the substituents R¹ and R² are as defined in claim 1; wherein the substituent ‘X’ is a halogen;
wherein the nucleophilic leaving group (NLG) is selected from the group consisting of a halogen group, a thio group, a cyano group, a tosylate group, an alkoxy group, an aryloxy group, an alkyl ester group, an azide group, and an amine group.

9. The method of claim 7, wherein the phosphazene compound (T) is a cyclic phosphazene compound represented by the structure: 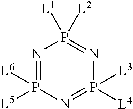
wherein the substituents L¹, L², L³, L⁴, L⁵ and L′ are independently the same or different, and are independently selected from the nucleophilic leaving group (NLG), a substituted or an unsubstituted alkyl group having 1-20 carbon atoms, a branched or a linear alkyl group having 1-20 carbon atoms, an alkenyl group having 1-20 carbon atoms, an aryl group having 6-30 carbon atoms, an alkaryl group having 6-30 carbon atoms, an alkenyl substituted aryl group having 6-30 carbon atoms, an aralkyl group having 6-30 carbon atoms, a cycloalkyl group having 3-10 carbon atoms, and a substituted or an unsubstituted amine group, with the proviso that at least one of the substituents L¹, L², L³, L⁴, L⁵ or L⁶ is a nucleophilic leaving group (NLG), all of the substituents L¹, L², L³, L⁴, L⁵ and L⁶ are a nucleophilic leaving group (NLG), each of the substituents L¹, L², L³, L⁴, L⁵ and L⁶ is a halogen, or each of the substituents L¹, L², L³, L⁴, L⁵ and L⁶ is chlorine.

10. The method of claim 7, wherein the phosphazene compound (T) is a cyclic phosphazene compound represented by the formula: 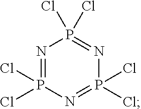 [SMILES: ClP1(Cl)=NP(Cl)(Cl)=NP(Cl)(Cl)=N1]
the diol compound (V) is represented by the formula: 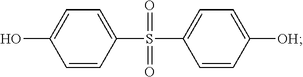 [SMILES: O=S(=O)(c1ccc(O)cc1)c1ccc(O)cc1]
and
the sulfonyl halide compound (W) is represented by the formula:

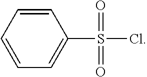

11. The method as claimed in claim 7, wherein the base is selected from KOH, NaOH, LiOH, NaH, KH, K₂CO₃, Na₂CO₃, LizCO₃, K₃PO₄, pyridine, 4-dimethylaminopyridine, trimethylamine, and triethylamine, and any combinations thereof.

12. A polymer composition comprising:
i. ≥80.0 wt. % and ≤99.95 wt. %, or ≥85.0 wt. % and ≤99.95 wt. %, with regard to the total weight of the composition, of a thermoplastic polymer; and
ii. ≥0.05 wt. % and ≤20.0 wt. %, of ≥0.05 wt. % and ≤15.0 wt. %, with regard to the total weight of the composition, of the sulfonate esterified phosphazene compound of claim 1.

13. The polymer composition of claim 12, wherein the thermoplastic polymer is selected from polyethylene terephthalate (PET), impact polypropylene copolymers, propylene ethylene copolymers, propylene ethylene alpha olefin terpolymers, polycarbonate (PC), polybutylene terephthalate (PBT), poly(1,4-cyclohexylidene cyclohexane-1,4-dicarboxylate) (PCCD), glycol modified polycyclohexyl terephthalate (PCTG), poly(phenylene oxide) (PPO), polypropylene (PP), polyethylene (PE), polyvinyl chloride (PVC), ethylene-C₄-C₁₂-alpha-olefin copolymers, polystyrene (PS), polymethyl methacrylate (PMMA), polyethyleneimine or polyetherimide (PEI) and their derivatives, thermoplastic elastomer (TPE), terephthalic acid (TPA) elastomers, poly(cyclohexanedimethylene terephthalate) (PCT), polyethylene naphthalate (PEN), polyamide (PA), polysulfone sulfonate (PSS), sulfonates of polysulfones, polyether ether ketone (PEEK), acrylonitrile butyldiene styrene (ABS), polyether ketone ketone (PEKK), and polyphenylene sulfide (PPS), co-polymers thereof, and blends thereof.

14. An article comprising the polymer composition of claim 12.

15. A method of improving the fire retardant properties of a polymer composition, comprising adding the sulfonate esterified phosphazene compound of claim 1 to the polymer composition.

16. The method of claim 7, wherein the substituent ‘X’ is chlorine and the nucleophilic leaving group (NLG) is a halogen group.

17. The method of claim 8, wherein the substituent ‘X’ is chlorine and the nucleophilic leaving group (NLG) is a halogen group.

18. The method of claim 8, wherein the phosphazene compound (T) is a cyclic phosphazene compound represented by the structure: 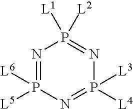
wherein the substituents L¹, L², L³, L⁴, L⁵ and L⁶ are independently the same or different, and are independently selected from the nucleophilic leaving group (NLG), a substituted or an unsubstituted alkyl group having 1-20 carbon atoms, a branched or a linear alkyl group having 1-20 carbon atoms, an alkenyl group having 1-20 carbon atoms, an aryl group having 6-30 carbon atoms, an alkaryl group having 6-30 carbon atoms, an alkenyl substituted aryl group having 6-30 carbon atoms, an aralkyl group having 6-30 carbon atoms, a cycloalkyl group having 3-10 carbon atoms, and a substituted or an unsubstituted amine group, with the proviso that at least one of the substituents L¹, L², L³, L⁴, L⁵ or L⁶ is a nucleophilic leaving group (NLG), all of the substituents L¹, L², L³, L⁴, L⁵ and L⁶ are a nucleophilic leaving group (NLG), each of the substituents L¹, L², L³, L⁴, L′ and L⁶ is a halogen, or each of the substituents L¹, L², L³, L⁴, L⁵ and L⁶ is chlorine.

19. The method of claim 8, wherein the phosphazene compound (T) is a cyclic phosphazene compound represented by the formula: 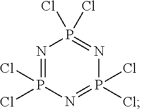 [SMILES: ClP1(Cl)=NP(Cl)(Cl)=NP(Cl)(Cl)=N1]
the diol compound (V) is represented by the formula: 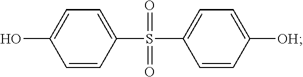 [SMILES: O=S(=O)(c1ccc(O)cc1)c1ccc(O)cc1]
and the sulfonyl halide compound (W) is represented by the formula:

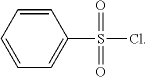

20. The method of claim 8, wherein the base is selected from KOH, NaOH, LiOH, NaH, KH, K₂CO₃, Na₂CO₃, LizCO₃, K₃PO₄, pyridine, 4-dimethylaminopyridine, trimethylamine, and triethylamine, and any combinations thereof.
